(19) United States
(12) Patent Application Publication
(10) Pub. No.: US 2025/0251397 A1
(43) Pub. Date: Aug. 7, 2025

(54) METHOD FOR PERFORMING EARLY DIAGNOSIS OF SEPSIS USING ANALYSIS DEVICE INCLUDING MICRO FILTER

(71) Applicant: GLORY BIOTECH CORP., Seoul (KR)

(72) Inventors: Sung Yeon Moon, Seoul (KR); Lee Jae Young, Seoul (KR); Back Seung Lim, Goyang-si (KR); Aghamoradi Shabnam, Seoul (KR)

(73) Assignee: GLORY BIOTECH CORP., Seoul (KR)

(21) Appl. No.: 19/042,493

(22) Filed: Jan. 31, 2025

(30) Foreign Application Priority Data
Feb. 2, 2024 (KR) 10-2024-0016443
May 13, 2024 (KR) 10-2024-0062316

(51) Int. Cl. G01N 33/569; G01N 33/74

(52) U.S. Cl. CPC G01N 33/56972; G01N 33/74; G01N 2333/585; G01N 2333/70535

(57) ABSTRACT

Provided is a method for performing early diagnosis of sepsis using an analysis device including a micro filter. The method includes an operation of forming, in a blood sample to be analyzed, neutrophil Cluster of Differentiation (CD)64 cells to which a fluorescent material is bound and a procalcitonin (PCT) polymer to which a fluorescent material is bound and beads are applied; an operation of simultaneously capturing, by the micro filter, neutrophil CD64 cells and a PCT polymer from the blood sample; an operation of radiating, by the light source part, light toward the micro filter; an operation of detecting, by the photosensitive part, light passing through the micro filter; an operation of outputting, by the display part, information acquired using the photosensitive part; and an operation of analyzing, by the analysis part, an image displayed on the display part to determine whether there is sepsis.

### CROSS-REFERENCE TO RELATED APPLICATIONS

This application claims priority to Korean Patent Application No. 10-2024-0016443 filed on Feb. 2, 2024 and Korean Patent Application No. 10-2024-0062316 filed on May 13, 2024, the entire contents of which are herein incorporated by reference.

### 1. FIELD

The present disclosure relates to a method for performing early diagnosis of sepsis using an analysis device including a micro filter, and more particularly to a method for performing early diagnosis of sepsis which is capable of diagnosing sepsis early and reliably using an analysis device including a micro filter configured to capture neutrophil Cluster of Differentiation (CD)64 cells and procalcitonin (PCT) polymers. Throughout this specification, PCT is an abbreviation for procalcitonin.

### 2. DESCRIPTION OF THE RELATED ART

Sepsis is a disease in which, when the blood is infected due to bacteria or the like which have invaded the human body, and clinically, an inflammatory response such as increased body temperature, heart rate, respiratory rate, and white blood cell count appears throughout the body. In addition, pneumonia which has become a topic of increasing interest due to coronavirus disease (COVID)-19 is a fatal disease for the elderly and other vulnerable people. If an elderly person catches pneumonia, it is known that sepsis caused due to pneumonia, rather than pneumonia itself, is the main cause of death in the elderly person who catches pneumonia.

In this way, although sepsis is a particularly fatal disease in the elderly people with a weak or compromised immune system, due to the inaccuracy of diagnostic methods, long time to confirm results, rapid progression when the disease occurs, a high mortality rate, or the like, this is a disease which has become huge matters in hospitals, particularly emergency rooms, but it is difficult to respond appropriately on site due to the difficulty in rapid and accurate diagnosis.

Particularly, even in the case of early sepsis, the mortality rate is known to be 30 to 40% and the prognosis is poor if symptoms occur within 2 hours of onset or at the latest after a day, and thus early detection is essential.

Here, although commonly used biomarkers for blood infections include C-reactive proteins (CRPs), procalcitonins (PCTs), tumor necrosis factors (TNFs), interlukin (IL)-6, and the like, most biomarkers have non-specific diagnostic properties, making results thereof unreliable. For this reason, the diagnosis of sepsis to date has mostly relied on clinical determination based on the experience of medical staff.

### SUMMARY

The present disclosure was created to solve the above matters, and the present disclosure is for the purpose of providing a method for performing early diagnosis of sepsis which is capable of diagnosing sepsis reliably at an early stage.

A sepsis diagnosis method according to an example embodiment of the present disclosure is a method for performing early diagnosis of sepsis using an analysis device including a micro filter having micro filtration holes, the method for performing early diagnosis of sepsis in which the analysis device includes the micro filter, a light source part which radiates light directly toward the micro filter, a photosensitive part which detects light passing through the micro filter, a display part which outputs information acquired using the photosensitive part, and an analysis part which analyzes information from an image displayed on the display part to determine whether there is sepsis, the method for performing early diagnosis of sepsis include an operation of forming, in a blood sample to be analyzed, neutrophil Cluster of Differentiation (CD)64 cells to which a fluorescent material is bound and a procalcitonin (PCT) polymer to which a fluorescent material is bound and beads are applied; an operation of simultaneously capturing, by the micro filter, neutrophil CD64 cells and a PCT polymer from the blood sample; an operation of radiating, by the light source part, light toward the micro filter; an operation of detecting, by the photosensitive part, light passing through the micro filter; an operation of outputting, by the display part, information acquired using the photosensitive part; and an operation of analyzing, by the analysis part, an image displayed on the display part to determine whether there is sepsis, the operation of determining whether there is sepsis includes performing a determination concerning whether a disease is bacterial sepsis, through analysis of the displayed image, when the number of nCD64 cell individuals and PCT concentration are derived and the derived PCT concentration is 0.5 ng/mL or more and the derived number of neutrophil CD64 cell individuals is 1000 cells/μl or more, a size of the micro filtration holes is set so that the micro filtration holes capture the neutrophil CD64 cells, and a size of the beads is set so that a PCT polymer is captured using the micro filtration holes.

According to an example embodiment of the present disclosure, the operation of determining whether there is sepsis may include deriving, through analysis of the displayed image, the number of neutrophil CD64 cell individuals and PCT concentration and determining whether there is sepsis on the basis of the derived number of neutrophil CD64 cell individuals and PCT concentration.

According to an example embodiment of the present disclosure, the operation of determining whether there is sepsis includes determining that a disease is bacterial sepsis when the derived PCT concentration is a first prescribed value or more and the derived number of neutrophil CD64 cell individuals is a second prescribed value or more and determining that a disease is viral sepsis when the derived PCT concentration is the first prescribed value or more and the derived number of neutrophil CD64 cell individuals is less than the second prescribed value.

According to an example embodiment of the present disclosure, the first prescribed value may be 0.5 ng/ml and the second prescribed value may be 1000 cells/μl.

According to an example embodiment of the present disclosure, the micro filter may include a filter cartridge and the filter cartridge may be configured to include a plurality of filtration holes having a size in which the filtration holes are capable of capturing the neutrophil CD64 cells and the PCT polymer.

According to an example embodiment of the present disclosure, neutrophil CD64 cells to which the fluorescent material is bound are produced by binding a detection antibody and a fluorescent material to cells in which a CD64 marker exists on a membrane and the PCT polymer may be produced by binding beads, capture antibodies, detection antibodies, PCT, and a fluorescent material.

According to an example embodiment of the present disclosure, neutrophil CD64 cells and PCT polymers bound to a fluorescent material for nCD64 and PCT which are one of the biomarkers are produced and applied to an analysis device including a micro filter to rapidly and reliably detect and monitor the number of neutrophil CD64 cell individuals and PCT concentration. Therefore, a faster and more reliable early diagnosis of sepsis is possible compared to sepsis diagnosis using biomarker tests or methods involving bacterial culture or sepsis diagnosis in the related art based on a clinical determination based on the experience of medical staff.

### BRIEF DESCRIPTION OF THE DRAWINGS

FIG. 1 is a schematic diagram of a target polymer according to an example embodiment of the present disclosure.

FIG. 2 shows a state in which antibodies and fluorescent substances are bound to an nCD64 marker on a cell membrane according to an example embodiment of the present disclosure.

FIG. 3 is a perspective view of a filter cartridge according to an example embodiment of the present disclosure.

FIG. 4 is a cross-sectional view of a filter cartridge according to an example embodiment of the present disclosure.

FIG. 5 is a schematic diagram of an analysis device using a micro filter according to an example embodiment of the present disclosure.

FIG. 6 shows a flow chart for describing a sepsis diagnosis method using an analysis device according to an example embodiment of the present disclosure.

FIG. 7 shows PCT polymers and neutrophil CD64 cells captured using an analysis device according to an example embodiment of the present disclosure, showing fluorescently labeled PCT polymers and fluorescently labeled neutrophil CD64 cells.

FIG. 8 shows a correlation relationship between actual PCT concentration and the number of PCT polymer individuals measured using an analysis device according to an example embodiment of the present disclosure.

FIG. 9 is a diagram for explaining a degree of likelihood of sepsis according to PCT concentration.

FIG. 10 shows a correlation relationship between the number of neutrophil CD64 cell individuals and PCT concentration obtained using an analysis device according to an example embodiment of the present disclosure and bacterial sepsis or viral sepsis.

### DETAILED DESCRIPTION

A preferred embodiment of the present disclosure will be described in detail below with reference to the attached drawings.

In order to clearly explain the invention, specific descriptions of parts unrelated to the present disclosure are omitted and the same reference numerals are used to describe the same constituent elements throughout the specification. Furthermore, the shape and the size of each constituent element illustrated in the drawings are arbitrarily illustrated for convenience of explanation, and thus the present disclosure is not necessarily limited to the illustrated shape and size. That is to say, it needs to be understood that specific shapes, structures, and characteristics described in the specification may be modified and implemented from an example embodiment to another example embodiment without departing from the spirit and the scope of the present disclosure and that the positions or the dispositions of individual constituent elements may also be changed without departing from the spirit and the scope of the present disclosure. Therefore, the detailed description which will be set forth below is not intended to be limiting and the scope of the present disclosure needs to be accepted as encompassing the scope claimed in the claims of the patent and all scopes equivalent thereto.

Throughout the specification, whenever it is said that a part “includes” a constituent element, this does not mean that it excludes other constituent elements, but rather that it may include other constituent elements, unless otherwise specifically stated.

Parts designated by the same reference number throughout the specification represent the same or similar constituent elements.

The present disclosure relates to a method for performing early diagnosis of sepsis using an analysis device including a micro filter, and the present disclosure is for the purpose of diagnosing sepsis early and reliably by early testing and monitoring two or more biomarkers including neutrophil Cluster of Differentiation (nCD)64 cells and a procalcitonin (PCT) polymer.

To this end, in the present disclosure, sepsis is diagnosed using an analysis device including a micro filter as will be described below to quickly and reliably detect the number of nCD64 and PCT individuals. The production of neutrophil CD64 cells and PCT polymers having a fluorescent material bound thereto will be first explained, followed by the examination and the monitoring of nCD64 and PCT using micro filters will be explained.

### 1. Regarding Production of Neutrophil CD64 Cells and PCT Polymers Bound to Fluorescent Material

The key to producing neutrophil CD64 cells and PCT polymers bound to a fluorescent material according to an example embodiment of the present disclosure is to produce neutrophil CD64 cells and PCT polymers bound to a fluorescent material which is capable of being filtered by applying a micro filter, as will be described below.

As will be described below, the present disclosure uses, at least in a part thereof, a fluorescence immunoassay. Fluorescence immunoassay measures the binding of antigens and antibodies using fluorescence. Fluorescence is a reaction in which a fluorescent material absorbs light of a specific wavelength, the molecules of the fluorescent material are excited and then return to an original state thereof, and light of a different wavelength than the light which has been absorbed is emitted. For example, a fluorescent material is attached to an antibody which reacts to the antigen to be measured to induce an antigen-antibody reaction. After the reaction has occurred, if light of a wavelength which is capable of causing fluorescence is projected, fluorescence is emitted in proportion to an amount of fluorescent material and the concentration of the antigen may be calculated from the amount of this fluorescence. The production of PCT polymers and neutrophil CD64 antibodies bound to a fluorescent material will be described below.

### A. Production of PCT Polymers Bound to Fluorescent Material

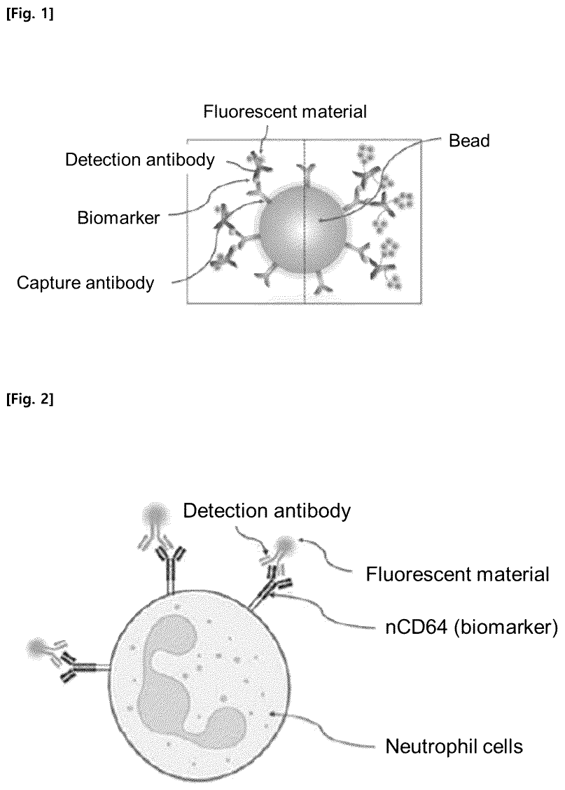

Since a size of PCT itself is very small, in the present disclosure, beads in the form of microspheres of a prescribed size may be applied to produce polymers of a prescribed size so that a micro filter which will be described later may be applied. Furthermore, as antibodies, a detection antibody which binds to a specific antigen (biomarker) and to which a prescribed fluorescent material is attached for detection and a captured antibody which binds to the specific antigen and to fix the specific antigen on a bead may be used. FIG. 1 is a schematic diagram of PCT polymers according to an example embodiment of the present disclosure.

As shown in FIG. 1, the PCT polymers may be produced by sequentially binding captured antibodies, biomarkers (PCT), detection antibodies, and fluorescent materials to beads. Thus, the PCT polymers have a size in which it may be separated when applied to a micro filter while bound to a fluorescent material. The present disclosure is for measuring PCT as a biomarker and the antigen shown in FIG. 1 may be PCT.

### B. Production of Neutrophil CD64 Antibody Bound to Fluorescent Material

nCD64 is a CD marker which is present on the membrane of neutrophil cells, not in a free polymer state.

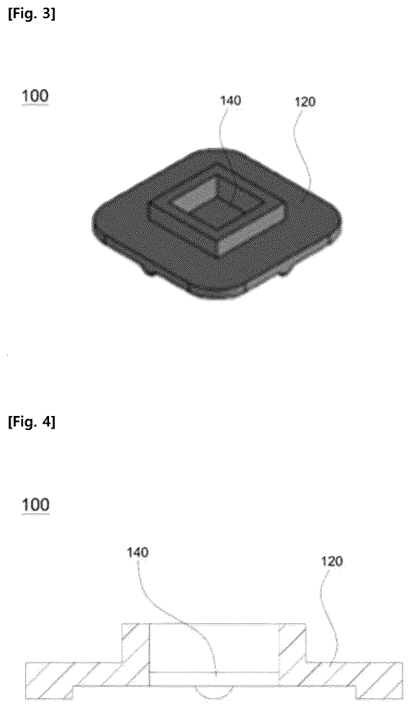

FIG. 2 shows a state in which an antibody, a fluorescent material, is bound to the nCD64 marker on the membrane of neutrophil cells (through an antigen-antibody reaction) according to an example embodiment of the present disclosure.

As shown in FIG. 2, unlike in the case of PCT, since the nCD64 marker exists on the membrane of neutrophil cells of the prescribed size, by sequentially binding the detection antibody and the fluorescent material to the nCD64 marker on the membrane of neutrophil cells, a micro filter suitable for separating neutrophil cells may be applied to separate neutrophil cells to which the fluorescent material is bound.

### 2. Regarding Micro Filter

A micro filter according to an example embodiment of the present disclosure may be configured to include a filter cartridge for capturing PCT polymers and neutrophil cells. In the present disclosure, other configurations of the filter, excluding the filter cartridge, be implemented as a typical filter configuration and are not limited to a specific filter configuration. Thus, a description of other configurations of the filter is omitted.

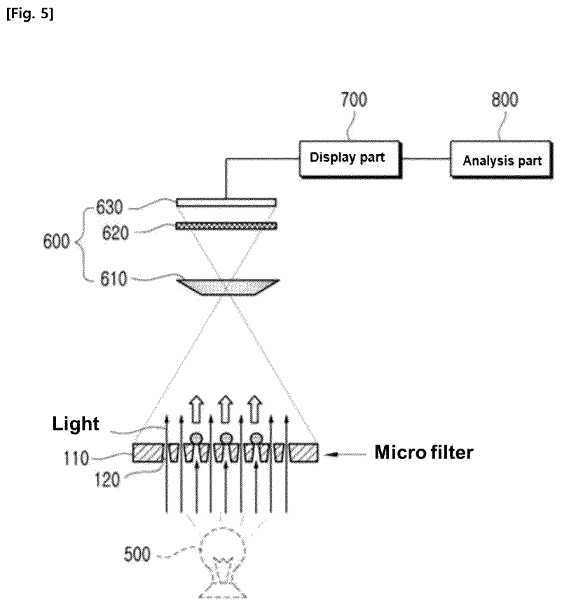

FIG. 3 is a perspective view of a filter cartridge according to an example embodiment of the present disclosure. FIG. 4 is a cross-sectional view of the filter cartridge shown in FIG. 3.

As shown in FIGS. 3 and 4, a filter cartridge 100 may be composed of a filter cartridge main body 120 which may be detachably installed in a micro filter and a filtration filter 140 which is attached to the filter cartridge main body. A plurality of micro filtration holes (not shown) are formed in the filtration filter 140. For convenience of use, such a filter cartridge 100 may be applied in a disposable manner by being installed in a micro filter and removed after use.

A plurality of filter holes may be formed to have a circular shape and a diameter of circular filtration holes may be 3 to 5 μm, considering a size of PCT polymers and a size of neutrophil cells.

If the diameter of the filtration holes is larger than this, the PCT polymers or neutrophil cells may not be properly captured, resulting in a decrease in the recovery rate of PCT polymers or neutrophil cells included in a sample blood.

Also, a center interval between any one filtration hole and any adjacent other filtration holes is preferably 10 μm to 27 μm. When a separated distance is too short, images of the filtration holes may overlap in an image displayed on a display part 700 which will be described later, and if it is too long, the number of filtration hole individuals to capture a target object may not be sufficient.

### 3. Regarding Analysis Device Using Micro Filter

An analysis device using a micro filter according to an embodiment of the present disclosure will be described below.

FIG. 5 is a schematic diagram of an analysis device using a micro filter according to an example embodiment of the present disclosure.

Referring to FIG. 5, the analysis device according to an example embodiment of the present disclosure includes a micro filter according to an example embodiment of the present disclosure, a light source part 500 which is located below the micro filter and radiates light toward the micro filter, and a photosensitive part 600 which is disposed to face the light source part 500 so that the micro filter is disposed between the light source part 500 and the photosensitive part 600 and detects light passing through the micro filter.

As described above, the micro filter captures PCT polymers or neutrophil cells through the filter cartridge described above.

The light source part 500 radiates light toward the micro filter. The target object (neutrophil CD64 cells, PCT polymers) captured by the micro filter is bound to a fluorescently labeled antibody. At this time, the fluorescent material absorbs light of a specific wavelength and becomes in an excited state. Since white light includes light of all wavelengths within the visible light range, it may be made to have this state, regardless of the type of fluorescent material. Thus, it is preferable that the light source part 500 be an LED, a mercury lamp, or a halogen lamp which radiates white light.

The photosensitive part 600 detects light passing through the micro filter. The photosensitive part 600 includes a lens 610 which is located below an excitation light filter 620, the excitation light filter 620 which is located above the lens 610, and an image sensor 630 which is located above the excitation light filter 620.

The lens 610 converts light passing through the micro filter in the form of parallel light. It is preferable that the lens 610 be an optical component which collimates light. By converting light in the form of parallel light, the light in the form of parallel light reaches the image sensor 630, making image observation easier.

The excitation light filter 620 allows only light of a specific wavelength to pass therethrough. The fluorescent material labeled in the antibody has an excited state after absorbing a specific wavelength of light radiated by the light source part 500. At this time, it returns to a ground state again and emits wavelengths of a specific range. The excitation light filter 620 allows only wavelengths of a specific range to pass therethrough and blocks all other wavelengths, allowing only fluorescently labeled target objects to be observed.

The image sensor 630 detects light passing through the excitation light filter 620 and converts it into an electrical signal to form a digital image. Thus, light passing through the micro filter is converted into a digital image. It is preferable that the image sensor 630 be a CMOS sensor.

The analysis device according to an example embodiment of the present disclosure further includes the display part 700 which outputs information acquired using the photosensitive part 600 and an analysis part 800 which analyzes information from an image displayed on the display part 700.

The display part 700 displays a digital image converted in the image sensor 630. Thus, it is possible to visually check fluorescently labeled target objects (neutrophil CD64 cells, PCT polymers).

The analysis part 800 analyzes an image displayed on the display part 700. Information derived through analysis of the displayed image may include the number of neutrophil CD64 cell individuals, PCT concentration, whether a disease is likely to be bacterial sepsis, and whether a disease is likely to be viral sepsis.

The display part 700 may further display information derived using the analysis part 800. Thus, it is possible to visually check the number of neutrophil CD64 cell individuals, PCT polymer concentration, whether a disease is likely to be bacterial sepsis, and whether a disease is likely to be viral sepsis.

The display part 700 includes a notification part and the notification part may be configured to operate audibly in accordance with information analyzed and derived using the analysis part 800.

An analysis process of deriving, by the analysis part 800, the number of neutrophil CD64 cell individuals, PCT concentration, whether a disease is likely to be bacterial sepsis, and whether a disease is likely to be viral sepsis is explained in more details in “5. Analysis process of analysis part” which will be described below.

### 4. Regarding Sepsis Diagnosis Method Using Analysis Device

A sepsis diagnosis method using an analysis device according to an example embodiment of the present disclosure will be described below.

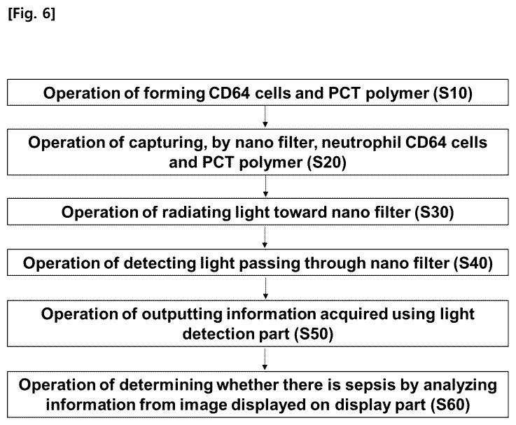

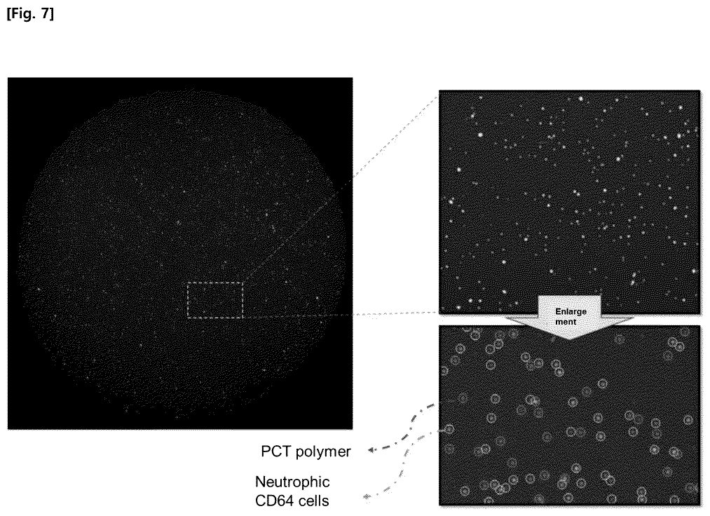

FIG. 6 shows a flow chart for describing a sepsis diagnosis method using an analysis device according to an example embodiment of the present disclosure.

Referring to FIG. 6, the sepsis diagnosis method according to an example embodiment of the present disclosure may include Operation S10 of forming neutrophil CD64 cells and a PCT polymer in a blood sample to be analyzed, Operation S20 of capturing, by the micro filter, the neutrophil CD64 cells and the PCT polymer (in a state in which fluorescent material is bound) from the blood sample, Operation S30 of radiating, by the light source part, light toward the micro filter, Operation S40 of detecting, by the photosensitive part, light passing through the micro filter, Operation S50 of outputting, by the display part, information acquired using the photosensitive part, and Operation S60 of analyzing, by the analysis part, an image displayed on the display part to perform a determination concerning whether there is sepsis.

Operation S10 of forming neutrophil CD64 cells and a PCT polymer bound to a fluorescent material in the blood sample to be analyzed is an operation of processing the blood sample before applying the blood sample to the micro filter. A PCT polymer and neutrophil CD64 cells bound to a fluorescent material may be produced as shown in FIG. 1 and FIG. 2 as an example. In addition, such a production process may be performed using a polymer production process or a fluorescent material treatment process in the related art. Thus, a detailed description thereof is omitted. At the time of producing a target object (neutrophil CD64 cells and a PCT polymer), the target object is fluorescently labeled with a fluorescent material. At this time, there may be at least one or more types of target objects. That is to say, it may be one of neutrophil CD64 cells and PCT polymers or it may be two types of neutrophil CD64 cells and PCT polymers. That is to say, it is possible to visually observe each of neutrophil CD64 cells and PCT polymers or to visually observe two types of target objects simultaneously.

Operation S20 of capturing, by the micro filter, neutrophil CD64 cells and PCT polymers from the blood sample may be performed, for example, by capturing neutrophil CD64 cells and PCT polymers through a filter cartridge of the micro filter. This has been explained in detail before and is omitted herein.

In Operation S30 of radiating, by the light source part, light toward the micro filter, as light is radiated, the fluorescent material labeled on the antibody absorbs light of a specific wavelength and has an excited state.

In relation to Operation S40 of detecting, by the photosensitive part 600, light passing through the micro filter, the photosensitive part 600 includes the lens 610, the excitation light filter 620, and an image sensor 630. The lens 610 converts light passing through a micro filter into parallel light, making image observation easier. Light in the form of parallel light passing through passes through the excitation light filter 620.

The excitation light filter 620 allows only wavelengths of a specific range to pass therethrough and the fluorescent material which absorbs the light radiated from the light source part 500 emits wavelengths of a specific range as it returns from the excited state to the ground state. The excitation light filter 620 changes a wavelength range in which it passes therethrough in accordance with the type of fluorescent material, allowing only fluorescently labeled target objects to be observed.

The image sensor 630 detects light passing through the excitation light filter 620 and converts it into an electrical signal to form a digital image. Thus, light passing through the micro filter is converted into a digital image.

In relation to Operation S50 of outputting, by the display part, information acquired using the photosensitive part, it is possible to, by the display part 700, visually check the digital image converted using the photosensitive part 600. When two types of target objects are used, images of different colors may be simultaneously checked in accordance with the type of fluorescent material labeled on the antibody.

Operation S60 of analyzing, by the analysis part, an image displayed on the display part to perform a determine concerning whether there is sepsis may include an operation of analyzing the image displayed on the display part to derive the number of neutrophil CD64 cell individuals and PCT concentration and an operation of determining whether a disease is likely to be bacterial sepsis and whether a disease is likely to be viral sepsis on the basis of the derived number of neutrophil CD64 cell individuals and PCT concentration.

In addition, the display part 700 may further display the information derived from the analysis part 800. For example, the number of neutrophil CD64 cell individuals, PCT concentration, whether a disease is likely to be bacterial sepsis, and whether a disease is likely to be viral sepsis information may be visually displayed on the display part 700. Furthermore, the display part 700 includes a notification part so that whether a disease is likely to be bacterial sepsis or whether a disease is likely to be viral sepsis may be audibly checked through the notification part.

### 5. Regarding Analysis Process of Analysis Part

### A. Derivation of Number of Neutrophil CD64 Cell Individuals, Number of PCT Polymer Individuals, and PCT Concentration

The inventors of the present disclosure conducted a test for determining whether a target object may be measured using an analysis device according to an example embodiment of the present disclosure.

In the test, neutrophil CD64 cells and PCT polymers bound to a fluorescent material applied in the present disclosure were used. Here, for PCT polymers, silica beads with a size of approximately 6 μm were applied. As fluorescent materials, for PCT polymers, green fluorescence, fluorescein isothiocyanate (FITC), was used, and for neutrophil CD64 cells, yellow fluorescence, phycoerythrin (PE), was used. At this time, dual color imaging was used for detecting two types of target objects using the same excitation light and fluorescence of different wavelengths, separating them by fluorescent labeling with two types of target objects. For this purpose, the wavelength that the excitation light filter 620 passes through was set to a wavelength which may simultaneously observe green fluorescence and yellow fluorescence. First, neutrophil CD64 cells and PCT polymers bound to a fluorescent material were produced and these were captured on a micro filter using a filter cartridge with 3 μm filtration holes and observed using an analysis device according to an example embodiment in the present disclosure. As a result, it was possible to simultaneously observe the fluorescence labeled on neutrophil CD64 cells and PCT polymers through the display part 700.

FIG. 7 shows PCT polymers and neutrophil CD64 cells observed using the analysis device according to an example embodiment of the present disclosure, showing PCT polymers fluorescently labeled with fluorescein isothiocyanate (green) and neutrophil CD64 cells fluorescently labeled with phycoerythrin (yellow). Thus, the analysis part 800 may derive the number of individuals by counting each of fluorescently labeled PCT polymers and neutrophil CD64 cells.

Also, the inventors of the present disclosure performed a test concerning whether a valid PCT polymers concentration may be obtained from the number of PCT polymer individuals measured using the analysis device according to an example embodiment of the present disclosure.

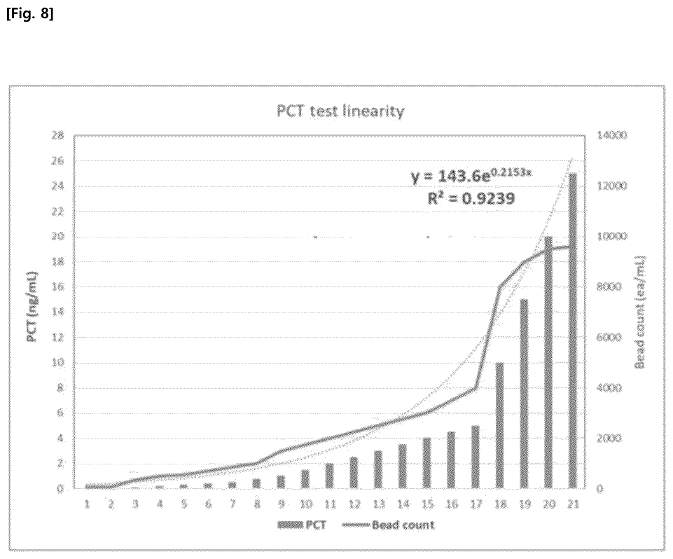

FIG. 8 shows a correlation relationship between the actual PCT concentration and the number of PCT polymer individuals obtained using the analysis device according to an example embodiment of the present disclosure as a result of the above test.

In the graph of FIG. 8, the bars represent the number of PCT polymer individuals (that is, the number of PCT polymer individuals derived using the analysis device according to an example embodiment of the present disclosure). In the graph of FIG. 8, the red solid line indicates the actual PCT concentration. The red dotted line in the graph of FIG. 8 represents the PCT concentration calculated on the basis of the number of PCT polymer individuals derived using the analysis device according to an example embodiment of the present disclosure. As shown in FIG. 8, a high correlation was obtained between the calculated PCT concentration and the actual PCT polymers concentration. From these results, it was confirmed that, using the analysis device according to an example embodiment of the present disclosure, (1) PCT polymers may be formed, (2) the PCT polymers may be filtered through a filter, (3) the number of PCT polymer individuals may be derived through dual color imaging, and (4) the PCT concentration may be calculated with a precision equivalent to the actual PCT concentration from the derived number of PCT polymer individuals.

### B. Diagnosis of Sepsis

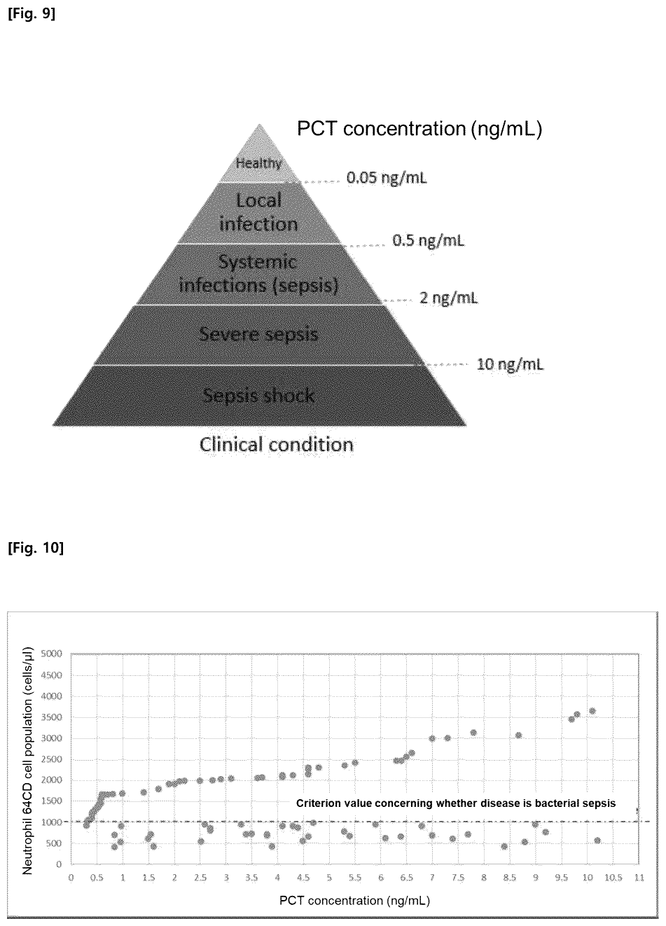

FIG. 9 is a diagram for explaining a degree of likelihood of sepsis according to PCT concentration. As shown in FIG. 9, when the PCT concentration is 0.5 ng/ml or higher, it is highly likely to be sepsis.

As described above, the analysis device according to an example embodiment of the present disclosure may form PCT polymers, capture them through a micro filter, derive the number of captured PCT polymer individuals, and calculate PCT concentration through this.

On the other hand, currently, sepsis is not diagnosed early and treated appropriately because it is not clear whether a patient has a bacterial or viral infection, and thus appropriate antibiotics may not be prescribed accordingly, and the mortality rate due to sepsis is increasing for this reason.

According to an example embodiment of the present disclosure, the analysis part 800 diagnoses whether a sample is bacterial or viral sepsis on the basis of the number of neutrophil CD64 cell individuals and PCT concentration (derived as described above).

The inventors of the present disclosure have verified a correlation relationship between the number of neutrophil CD64 cell individuals and PCT concentration and bacterial sepsis or viral sepsis, obtained using the analysis device according to an example embodiment of the present disclosure, through testing a plurality of bacterial sepsis or viral sepsis blood samples.

FIG. 10 shows a correlation relationship between the number of neutrophil CD64 cell individuals and PCT concentration and bacterial sepsis or viral sepsis obtained using the analysis device according to an example embodiment of the present disclosure.

The points marked in blue in the graph of FIG. 10 represent bacterial sepsis blood samples. The points marked in red in the graph of FIG. 9 represent viral sepsis blood samples.

As can be seen in FIG. 10, the bacterial sepsis blood sample showed that the number of neutrophil CD64 cell individuals was more than 1000 cells/μl and the viral sepsis blood sample showed that the number of neutrophil CD64 cell individuals was less than 1000 cells/μl. That is to say, it was verified that 1000 cells/μl may be the standard for distinguishing between bacterial sepsis and viral sepsis.

Based on these results, according to an example embodiment of the present disclosure, the analysis part 800 determines bacterial sepsis when the PCT concentration is 0.5 ng/ml or more and the number of neutrophil CD64 cell individuals is 1000 cells/μl or more. Furthermore, the analysis part 800 determines viral sepsis when the PCT concentration is 0.5 ng/ml or more and the number of neutrophil CD64 cell individuals is less than 1000 cells/μl.

When the analysis part 800 determines viral sepsis, the display part 700 may operate to visually indicate the likelihood of viral sepsis and the notification part may operate to audibly notify the likelihood of viral sepsis. Moreover, the analysis part 800 determines that it is bacterial sepsis, the display part 700 may visually display the possibility of bacterial sepsis and the notification part may operate to notify the likelihood of bacterial sepsis.

Although a preferred example embodiment of the present disclosure has been described above, the present disclosure is not limited to the above-described embodiment and includes all changes which are capable of being easily modified and deemed equivalent by a person having ordinary skill in the art to which the invention pertains from the embodiment of the present disclosure.

### EXPLANATIONS OF SYMBOLS

100: Filter cartridge
120: Filter cartridge main body
140: Filtration filter
500: Light source part
600: Photosensitive part
610: Lens
620: Excitation light filter
630: Image sensor
700: Display part
800: Analysis part

What is claimed is:

1. A method for performing early diagnosis of sepsis using an analysis device including a micro filter having micro filtration holes, wherein the analysis device includes the micro filter, a light source part which radiates light directly toward the micro filter, a photosensitive part which detects light passing through the micro filter, a display part which outputs information acquired using the photosensitive part, and an analysis part which analyzes information from an image displayed on the display part to determine whether there is sepsis,
the method for performing early diagnosis of sepsis comprises:
an operation of forming, in a blood sample to be analyzed, neutrophil Cluster of Differentiation (CD)64 cells to which a fluorescent material is bound and a procalcitonin (PCT) polymer to which a fluorescent material is bound and beads are applied;
an operation of simultaneously capturing, by the micro filter, neutrophil CD64 cells and a PCT polymer from the blood sample;
an operation of radiating, by the light source part, light toward the micro filter;
an operation of detecting, by the photosensitive part, light passing through the micro filter;
an operation of outputting, by the display part, information acquired using the photosensitive part; and
an operation of analyzing, by the analysis part, an image displayed on the display part to determine whether there is sepsis,
the operation of determining whether there is sepsis includes performing a determination concerning whether a disease is bacterial sepsis, through analysis of the displayed image, when the number of nCD64 cell individuals and PCT concentration are derived and the derived PCT concentration is 0.5 ng/ml or more and the derived number of neutrophil CD64 cell individuals is 1000 cells/μl or more,
a size of the micro filtration holes is set so that the micro filtration holes capture the neutrophil CD64 cells, and
a size of the beads is set so that a PCT polymer is captured using the micro filtration holes.

2. The method for performing early diagnosis of sepsis of claim 1, wherein the operation of determining whether there is sepsis includes
determining that a disease is viral sepsis when the derived PCT concentration is 0.5 ng/ml or more and the derived number of neutrophil CD64 cell individuals is less than 1000 cells/μl.

3. The method for performing early diagnosis of sepsis of claim 1, wherein the micro filter includes a filter cartridge, and
the filter cartridge is configured to include a plurality of filtration holes of a size in which the filtration holes are capable of capturing the neutrophil CD64 cells and the PCT polymer.

4. The method for performing early diagnosis of sepsis of claim 1, wherein neutrophil CD64 cells to which the fluorescent material is bound are produced by binding a detection antibody and a fluorescent material to neutrophil cells in which an nCD64 marker exists on a membrane, and
the PCT polymer is produced by binding beads, capture antibodies, detection antibodies, PCT, and a fluorescent material.
